(19) United States
(12) Patent Application Publication
(10) Pub. No.: US 2010/0087918 A1
(43) Pub. Date: Apr. 8, 2010

(54) CARDIOVASCULAR VALVE AND ASSEMBLY

(76) Inventors: Ivan Vesely, Larkspur, CO (US); Mark Mendel, Phoenix, AZ (US)

Correspondence Address: KUSNER & JAFFE;HIGHLAND PLACE SUITE 310, 6151 WILSON MILLS ROAD, HIGHLAND HEIGHTS, OH, 44143, US

(21) Appl. No.: 12/446,469

(22) PCT Filed: Oct. 18, 2007
(86) PCT No.: PCT/US07/22199

§ 371 Date: Apr. 21, 2009

Related U.S. Application Data
(60) Provisional application No. 60/862,475, filed on Oct. 23, 2006.

(51) Int. Cl. A61F 2/24

(52) U.S. Cl. 623/2.27

(57) ABSTRACT

A cardiovascular valve and assembly that facilitates valve installation and exchange. A mechanical cardiovascular valve having multiple sections and/or folding components provides easier valve installation. The cardiovascular valve assembly includes an exchangeable mechanical or bioprosthetic valve and a docking station to allow convenient replacement of a first valve with a second valve of the same or different type.

### RELATED APPLICATIONS

This application claims the benefit of U.S. Provisional Application No. 60/862,475, filed Oct. 23, 2006, which is fully incorporated herein by reference.

### FIELD OF INVENTION

The present invention relates generally to a cardiovascular valve and assembly, and more particularly to a cardiovascular valve and assembly that facilitate valve installation and valve exchange.

### BACKGROUND OF THE INVENTION

The demographics of patients suffering valvular disease are broad and the treatment modalities for each are complex. Historically, patients younger than 65 years of age have been prescribed mechanical heart valves, while older patients have been prescribed bioprosthetic heart valves that are comprised of biological tissue mounted on a plastic or metallic supporting structure. However, the role of the patient in choosing a particular valve type is changing. In this regard, younger patients that are active now frequently opt for bioprosthetic valves, since such patients are unwilling to deal with the lifestyle changes that are required by mechanical valves and the associated chronic anticoagulation therapy. These patients would much rather have repeat surgeries to replace a worn-out bioprosthetic valve, than deal with the lifestyle changes required by mechanical valves.

On the other hand, patients that that are afraid of repeat surgeries may opt for the mechanical valve. When that happens, the patient is destined to spend the rest of their life with the mechanical valve, even if they later decide that anticoagulation therapy is too restrictive. Such a patient may wish to change their mind, get off the anticoagulants and opt for a bioprosthetic valve at some time in the future. Furthermore, when a patient reaches an age of around 65 or 70, they may wish to opt for a bioprosthetic valve, since the durability of the bioprosthetic valve is expected to be 15-20 years. Mechanical valve patients may thus choose to have their mechanical valve exchanged for a bioprosthetic valve.

Conversely, bioprosthetic valve patients may choose to have their bioprosthetic valve exchanged for a mechanical valve, once they pass some critical phase of their life. For example, a young high school athlete may opt for a bioprosthetic valve so that the student may play competitive sports in school, but once the student becomes an adult and resumes a less physically demanding lifestyle, the individual may choose to get a mechanical valve when the bioprosthetic valve has worn out and needs replacement. With a mechanical valve, the individual will no longer need to undergo more valve procedures.

FIGS. 1 and 2 illustrate a prior art mechanical heart valve member 10. Valve member 10 is generally comprised of a substantially cylindrical valve body 12 defining a circular orifice or opening 20, and a pair of semi-circular occluders or leaflets 28A, 28B. Leaflets 28A and 28B are mounted within valve body 12 for pivoting between an open position to allow blood flow through opening 20 and a closed position to block blood flow. Leaflets 28A, 28B are mounted in valve body 12 by suitable interengagement means. The interengagement means may include depressions and/or protuberances on the interior of valve body 12 and cooperating depressions and/or protuberances at the periphery of each leaflet 28A, 28B. Valve member 10 shown in FIGS. 1 and 2 includes depressions formed within a pair of opposing extended wall portions 22 of valve body 12 and protuberances formed at the periphery of each leaflet 28A, 28B. A conventional sewing ring or cuff 30 is attached to the outer surface of valve body 12 for permanent attachment of valve member 10 to the tissue of the heart. Valve body 12 and leaflets 28A, 28B are typically formed of such materials as pyrolytic carbon. It should be appreciated that the large dimensions of valve member 10 can make it difficult to locate valve member 10 to the surgical site during a surgical implantation.

The present invention address the issues discussed above with respect to existing cardiovascular valves, and provides a permanent mechanical cardiovascular valve that is easier to locate to a surgical site, mechanical and bioprosthetic cardiovascular valves that are exchangeable, and a docking station (also referred to herein as a “base member”) that is adapted for use with both exchangeable mechanical valves and exchangeable bioprosthetic valves.

### SUMMARY OF THE INVENTION

In accordance with one aspect of the present invention, there is provided a mechanical cardiovascular valve member comprising: a first valve body section; a second valve body section, wherein said first valve body section is rotatably mounted to said second valve body section, said valve member moveable between an unfolded position and a folded position; and at least one leaflet pivotally mounted to at least one of said first and second valve body sections, said at least one leaflet movable between an open position and closed position.

In accordance with another aspect of the present invention, there is provided a cardiovascular valve assembly comprising: (a) a base member attachable to a tissue, the base member including at least one mounting portion; and (b) a mechanical valve member comprising: a first valve body section; a second valve body section; at least one leaflet pivotally mounted to at least one of said first and second valve body sections, said at least one leaflet movable between an open position and closed position; and at least one coupling element engageable with said at least one mounting portion to allow said mechanical cardiovascular valve member to be coupled to and decoupled from said base member.

In accordance with still another aspect of the present invention, there is provided a cardiovascular valve assembly comprising: (a) a base member attachable to a tissue, the base member including at least one mounting portion; and (b) a bioprosthetic valve member including: a valve frame comprising at least one wireform section, and at least one coupling element engageable with said at least one mounting portion to allow said bioprosthetic valve member to be coupled to and decoupled from said base member; and at least one leaflet mounted to said at least one wireform section.

In accordance with yet another aspect of the present invention, there is provided a mechanical cardiovascular valve member comprising: a first valve body section; a first leaflet pivotally mounted to said first valve body section for movement between an open position and closed position; a second valve body section; and a second leaflet pivotally mounted to said first valve body section for movement between an open position and closed position, wherein said first and second valve body sections form a mechanical cardiovascular valve when located adjacent to each other.

An advantage of the present invention is the provision of a permanent mechanical valve that is easily located to a surgical site.

Another advantage of the present invention is the provision of a mechanical valve that is exchangeable.

Still another advantage of the present invention is the provision of a cardiovascular valve assembly for exchangeable valves that includes a base member adapted to receive both mechanical and bioprosthetic valves.

These and other advantages will become apparent from the following description of embodiments of the present invention taken together with the accompanying drawings and the appended claims.

### BRIEF DESCRIPTION OF THE DRAWINGS

The invention may take physical form in certain parts and arrangement of parts, an embodiment of which will be described in detail in the specification and illustrated in the accompanying drawings which form a part hereof, and wherein:

FIG. 1 is a top plan view of a conventional prior art mechanical valve;

FIG. 2 is a perspective view of the conventional mechanical valve shown in FIG. 1;

FIG. 3 is a perspective view of a multi-part mechanical valve according to an embodiment of the present;

FIG. 4 is a perspective view of a foldable mechanical valve member according to another embodiment of the present invention, wherein the mechanical valve member is shown in a closed valve position;

FIG. 5 is a perspective view of the foldable mechanical valve member shown in FIG. 3, wherein the mechanical valve member is shown in an open valve position;

FIG. 6 is an exploded view of the foldable mechanical valve member of FIG. 4;

FIGS. 7 and 8 show the foldable mechanical valve member of FIG. 4 in a folded position;

FIG. 9 is an exploded view of a valve assembly according to still another embodiment of the present invention, the valve assembly including an exchangeable mechanical valve member and a docking station;

FIG. 10 is a perspective view of the valve assembly of FIG. 9, wherein the valve assembly is fully assembled with the exchangeable mechanical valve member coupled to the docking station;

FIG. 11 is an exploded view of a valve assembly according to yet another embodiment of the present invention, the valve assembly including an exchangeable bioprosthetic valve member and a docking station;

FIG. 12 is a perspective view of a valve frame of the exchangeable bioprosthetic valve member shown in FIG. 11; and

FIG. 13 is a perspective view of the valve assembly of FIG. 11, wherein the valve assembly is fully assembled with the exchangeable bioprosthetic valve member coupled to the docking station.

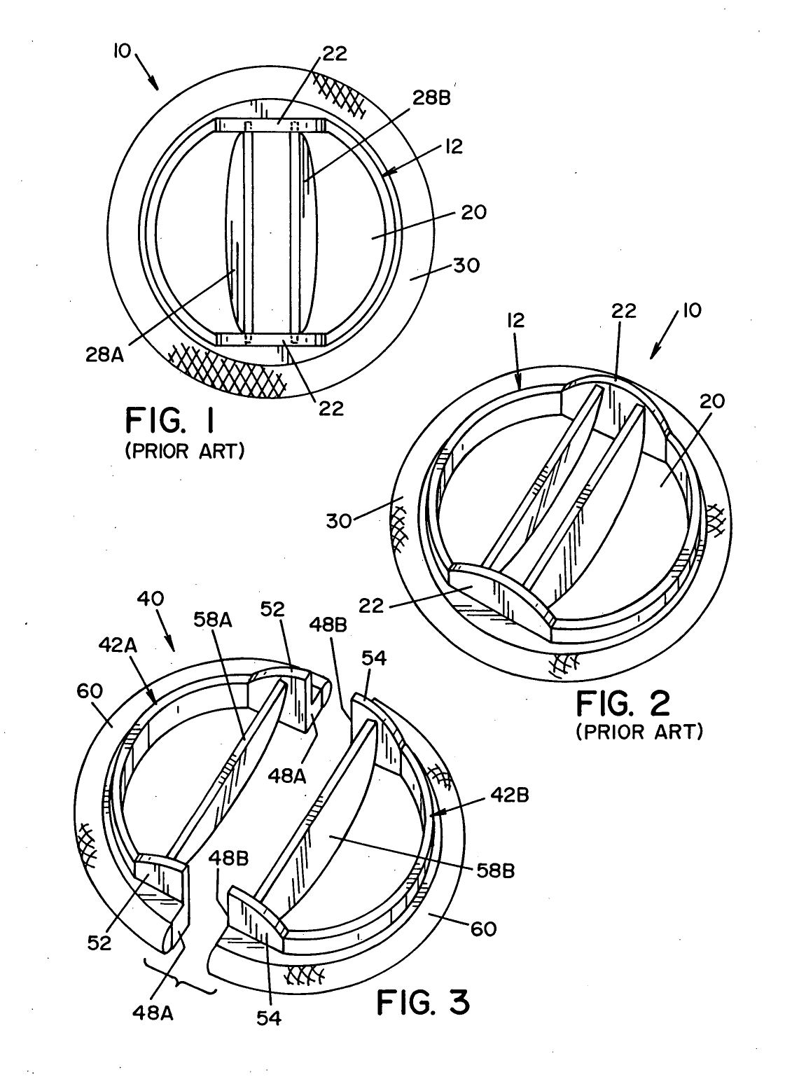

### DETAILED DESCRIPTION OF THE INVENTION

Referring now to the drawings wherein the showings are for the purpose of illustrating embodiments of the present invention only and not for the purposes of limiting same, FIG. 3 illustrates a multi-part mechanical cardiovascular valve member 40 according to a first embodiment of the present invention. Valve member 40 is generally comprised of a pair of substantially U-shaped valve body sections 42A and 42B, and a pair of semicircular leaflets 58A and 58B respectively mounted within valve body sections 42A, 42B for pivoting between an open position and a closed position. Leaflets 58A, 58B are respectively mounted in valve body sections 42A, 42B by suitable interengagement means that may include, but are not limited to, depressions and/or protuberances on the interior of valve body sections 42A and 42B and cooperating depressions and/or protuberances at the periphery of each leaflet 58A, 58B. In the illustrated embodiment, the depressions and/or protuberances of valve body sections 42A, 42B are respectively located on the interior of a pair of opposing extended wall portions 52 and 54 of valve body sections 42A, 42B. A substantially U-shaped sewing cuff or ring section 60 is attached to the outer surface of each valve body section 42A, 42B for permanent attachment of valve member 40 to the tissue of the heart. Valve body sections 42A, 42B and leaflets 58A, 58B are formed of suitable materials including, but not limited to, pyrolytic carbon, stainless steel, nitinol, polyurethane and other materials, as is well known in the mechanical valve field.

The two-part design of valve member 40 facilitates locating valve member 40 to a surgical site during a surgical implantation. In this respect, each valve body section 42A, 42B is individually located to the surgical site. Thus, each valve body section 42A, 42B can be individually located through an incision that is smaller than would be required for a fully assembled valve member 40.

At the surgical site, valve body sections 42A and 42B are located adjacent to each other such that respective front faces 48A and 48B are in engagement. A substantially circular orifice or opening is defined by adjacent valve body sections 42A and 42B. Sewing ring sections 60 attached to valve body sections 42A and 42B are used to secure each valve body section to the tissue of the heart.

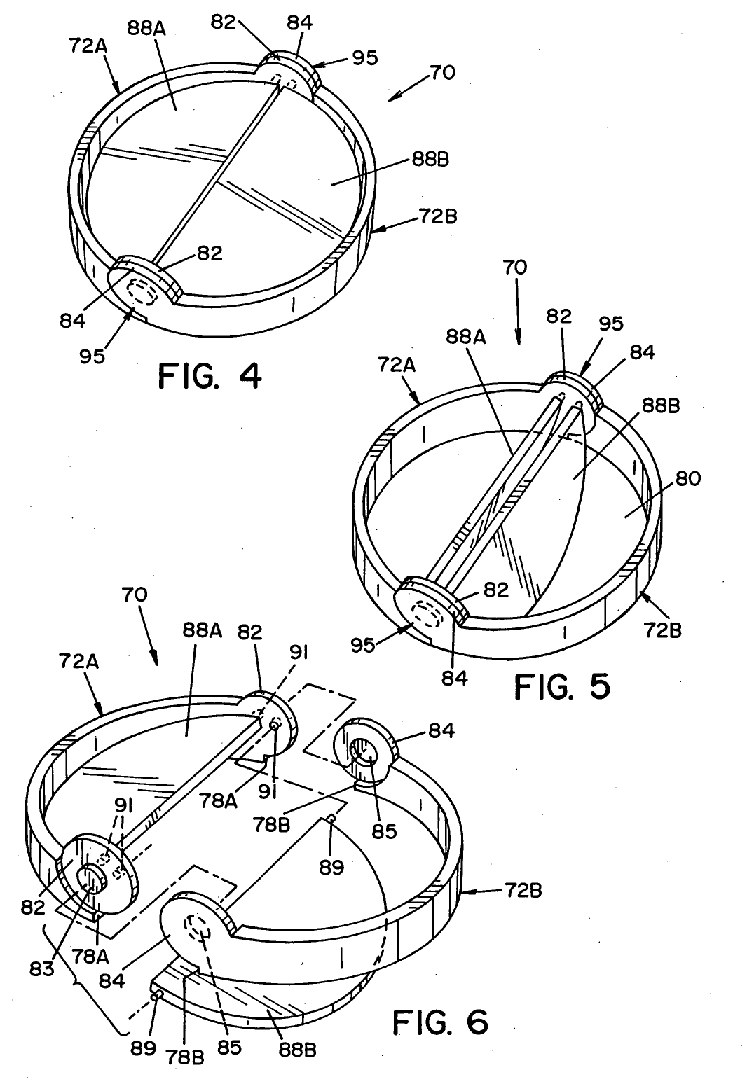

Referring now to FIGS. 4-8, there is shown a multi-part mechanical cardiovascular valve member 70 according to a second embodiment of the present invention. Valve member 70 is generally comprised of a pair of substantially U-shaped valve body sections 72A and 72B (as best seen in FIG. 6), and a pair of semicircular leaflets 88A and 88B, respectively located within valve body sections 72A, 72B, that pivot between a closed position (FIG. 4) and an open position (FIG. 5). Each valve body section 72A, 72B has a respective pair of opposing end wall portions 82, 84.

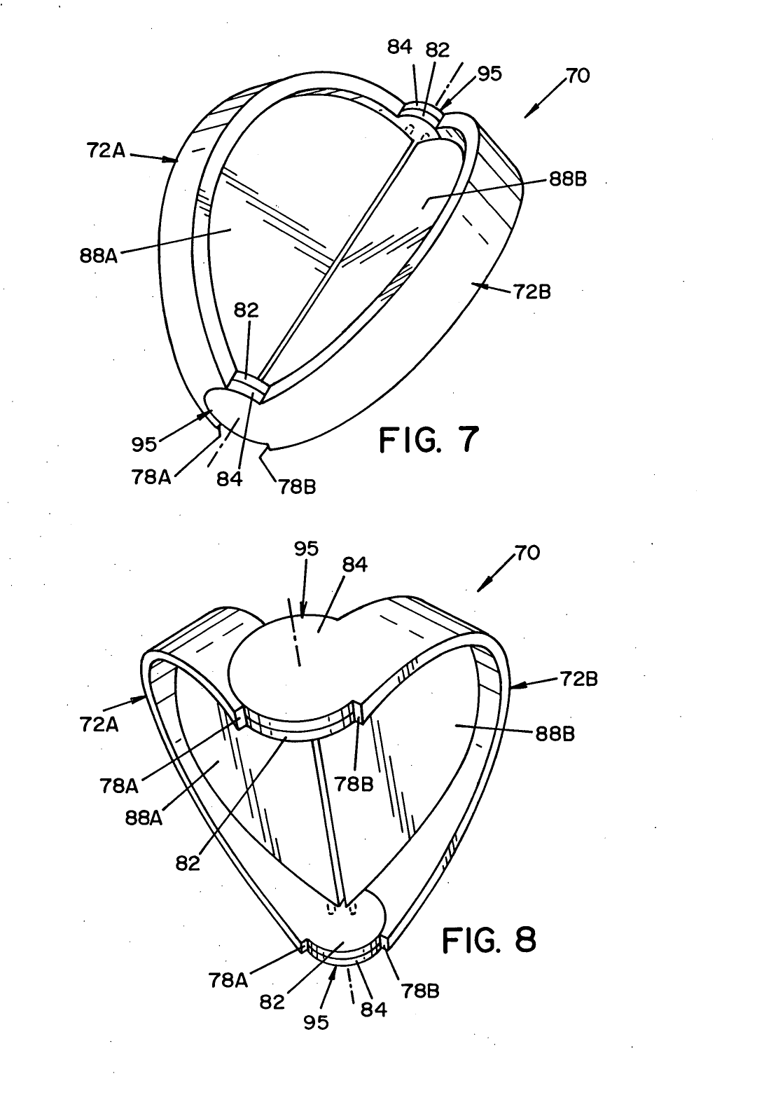

Valve body sections 72A and 72B are mounted to each other for pivoting valve member 70 between an unfolded position (FIGS. 4 and 5) and a folded position (FIGS. 7 and 8). In this regard, respective opposing end wall portions 82 and 84 of valve body sections 72A and 72B are rotatably mounted to each other by suitable interengagement means that may include, but are not limited to, cooperating depressions and/or protuberances on opposing end wall portions 82 and 84 of valve body sections 72A, 72B. In the illustrated embodiment, protuberances 83 are located on the exterior of opposing end wall portions 82 of valve body section 72A, and depressions 85 are located on the interior of opposing end wall portions 84 of valve body section 72B, as best seen in FIG. 6. The interengagement of end wall portions 82, 84 of valve body sections 72A, 72B form a pair of hinge portions 95 allowing rotational movement of valve body section 72A relative to valve body section 72B.

Leaflets 88A, 88B are located in valve body sections 72A, 72B by suitable interengagement means that may include, but are not limited to, depressions and/or protuberances on the interior of at least one of valve body sections 72A, 72B and cooperating depressions and/or protuberances at the periphery of each leaflet 88A, 88B. In the illustrated embodiment, both leaflets 88A and 88B are pivotally connected with valve body section 72A. In this respect, depressions 91 are located on the interior of the pair of opposing end wall portions 82 of valve body sections 72A, and protuberances 89 are located at the periphery of each leaflet 88A, 88B.

Valve body sections 72A, 72B and leaflets 88A, 88B are formed of suitable materials including, but not limited to, pyrolytic carbon, stainless steel, nitinol, polyurethane and other materials, as is well known in the mechanical valve field.

A substantially U-shaped sewing cuff or ring section (not shown) may be attached to the outer surface of each valve body section 72A, 72B for permanent attachment of valve member 70 to the tissue of the heart.

The folding design of valve member 70 facilitates locating valve member 70 to a surgical site during a surgical implantation. For example, valve member 70 is moved to the folded position to conveniently locate valve member 70 to the surgical site. Valve member 70 in a folded position can be located through an incision that is smaller than would be required for valve member 70 in the unfolded position.

At the surgical site, valve member 70 is moved to the unfolded position for appropriate installation. A substantially circular orifice or opening 80 (FIG. 5) is defined by valve body sections 72A, 72B, when valve member 70 is in the unfolded position. Respective front faces 78A and 78B of valve body sections 72A and 72B engage with each other when valve member 70 is fully unfolded and valve body sections 72A and 72B are substantially co-planar.

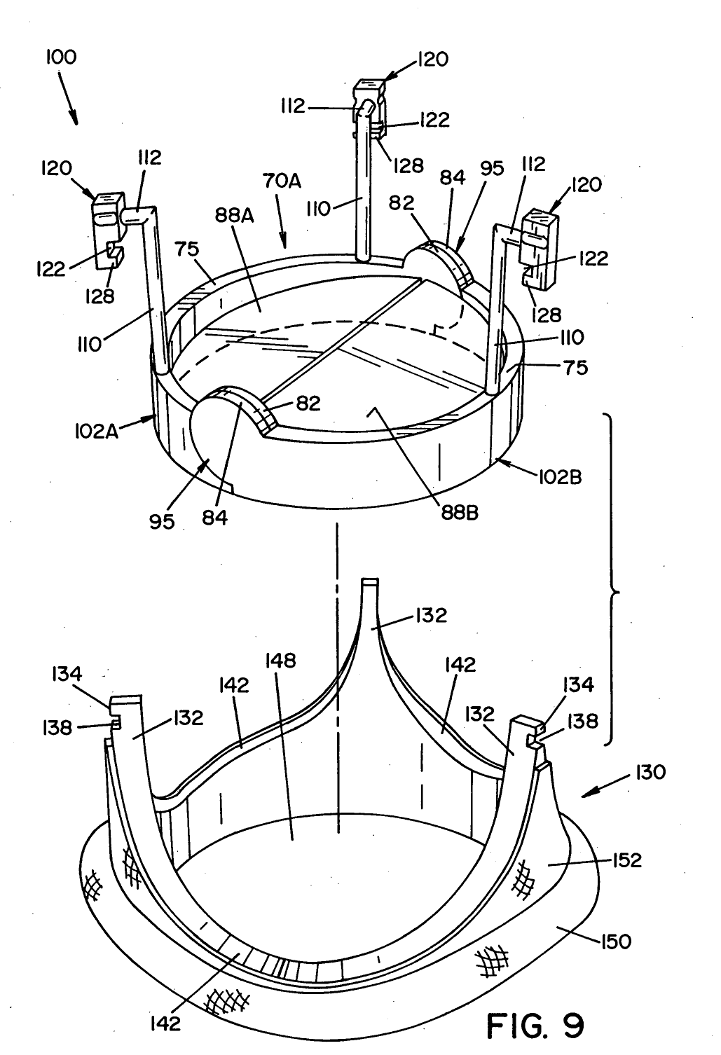

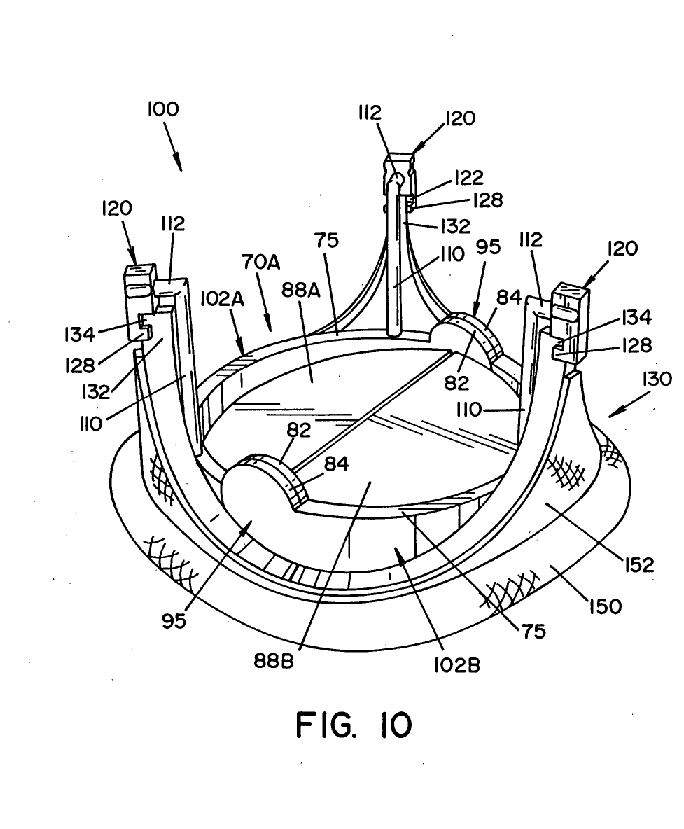

An alternative embodiment of multi-part mechanical cardiovascular valve member 70 will now be described. Referring now to FIGS. 9 and 10, there is shown a mechanical cardiovascular valve member 70A adapted for use as an exchangeable valve member in a cardiovascular valve assembly 100 comprised of valve member 70A and a docking station or base member 130.

Valve member 70A has substantially similar components as valve member 70 described in detail above. Accordingly, similar components bear the same reference numbers, and will not be described in detail. Like valve member 70, valve member 70A is generally comprised of a pair of substantially U-shaped valve body sections 102A and 102B, and a pair of semicircular leaflets 88A and 88B respectively located within valve body sections 102A, 102B for pivoting between an open position and a closed position. Valve body sections 102A and 102B are mounted to each other for pivoting valve member 70A between an unfolded position and a folded position. Valve member 70A also includes at least one L-shaped extension member 110 extending from top face 75 of each valve body section 102A, 102B. Each L-shaped extension member 110 includes an outward extending portion 112. A coupling element 120 is attached to each outward extending portion 112. Coupling element 120 includes an inward facing recess 122 and an inward extending tab 128. Coupling elements 120 allow valve member 70A to be coupled and uncoupled from base member 130, as will be described below.

Base member 130 is generally comprised of a plurality of mounting portions 132 and a plurality of arcuate sections 142 located between mounting portions 132. Each mounting portion 132 includes an outward extending tab 134 and a notch 138. Mounting portions 132 and arcuate sections 142 define a generally cylindrical recess 148 dimensioned to receive valve body sections 102A, 102B of valve member 70A. A sewing cuff or ring 150 is attached to the outer surface of base member 130 for permanent attachment of base member 130 to the tissue of the heart. Sewing ring 150 may also include a sleeve portion 152 to provide further coverage of the outer surface of base member 130.

Cardiovascular assembly 100 is installed by first attaching base member 130 to the tissue of the heart at a desired location. Thereafter, valve member 70A is installed by moving valve member 70A to the folded position and locating valve member 70A proximate to base member 130 at the surgical site. Valve member 70A is thereafter moved to the unfolded position.

As indicated above, coupling elements 120 allow valve member 70A to be coupled and uncoupled from base member 130. In this respect, recess 122 of coupling element 120 is dimensioned to receive tab 134 of mounting portion 132. Similarly, recess 138 of mounting portion 132 is dimensioned to receive tab 128 of coupling element 120. Valve member 70A is coupled and uncoupled from base 130 through engagement and disengagement of coupling element 120 and mounting portion 132. It should be understood that L-shaped extension member 110 of valve member 70A is formed of a material (e.g., medical grade steel, or pyrolitic carbon) having suitable elasticity to facilitate the engagement and disengagement of coupling element 120 and mounting portion 132. Cardiovascular assembly 100 is shown fully assembled in FIG. 10.

It should be appreciated that mechanical cardiovascular valve member 40 of FIG. 3 may be alternatively adapted for use as an exchangeable valve member in a cardiovascular valve assembly. For example, L-shaped extensions 110 and coupling elements 120 (or alternative interengagement means) may be added to valve member 40.

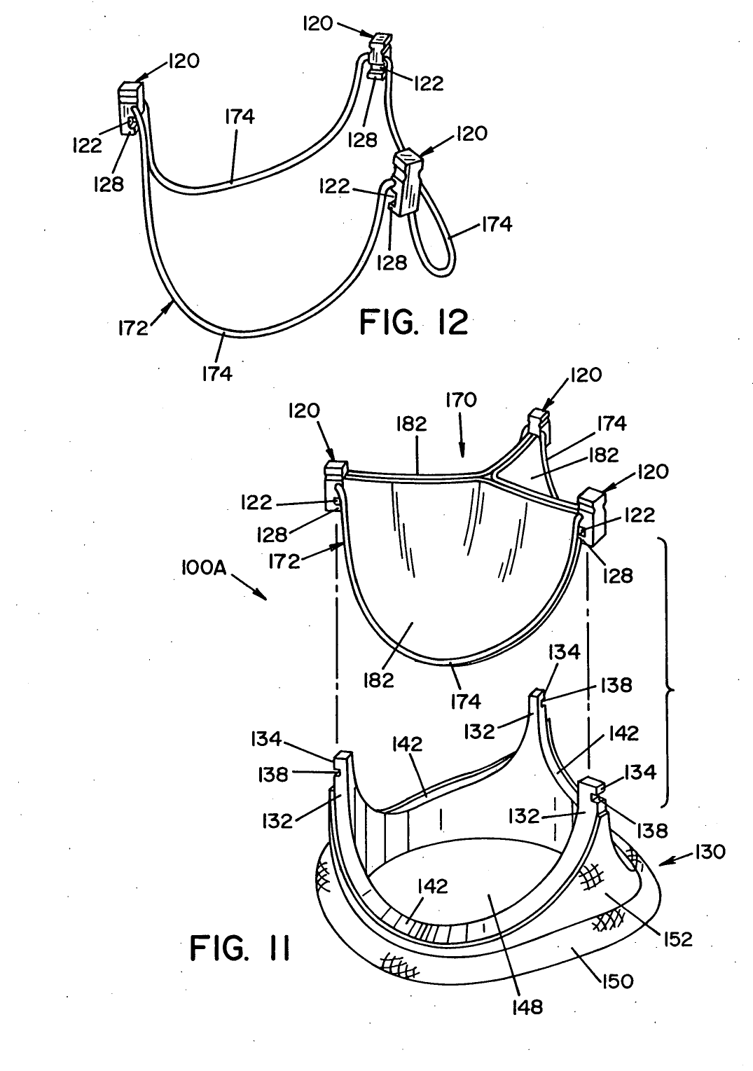

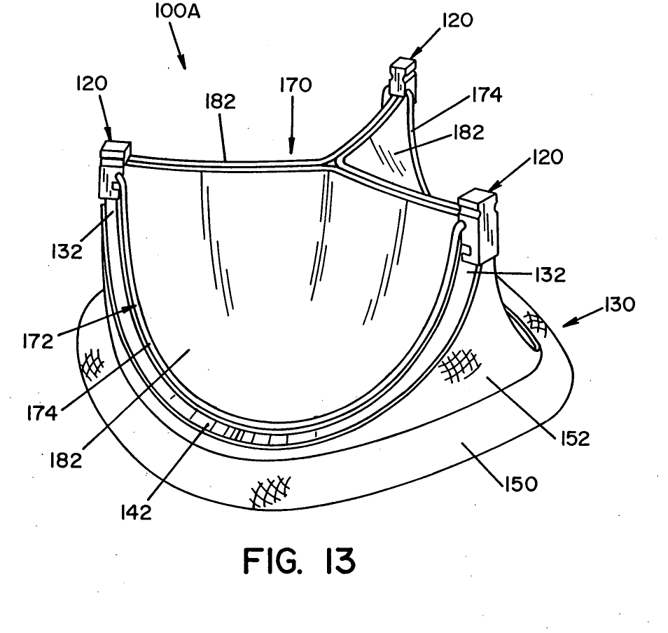

Referring now to FIGS. 11-13, there is shown a cardiovascular valve assembly 100A comprised of a bioprosthetic cardiovascular valve member 170 and docking station or base member 130 as described in detail above. Valve member 170 is generally comprised of a valve frame 172 (as best seen in FIG. 12), including a plurality of wireform sections 174 and coupling elements 120 (as described above), and one or more leaflets 182. Each wireform section 174 has a generally arcuate shape, and extends between coupling elements 120. Wireform sections 174 have an arcuate shape that matches the profile of arcuate sections 142 of base member 130, thereby forming a seal, as best seen in FIG. 13.

Wireform sections 174 are preferably made of a medical grade metal wire with suitable elasticity to facilitate the engagement and disengagement of coupling element 120 and mounting portion 132. Suitable materials include, but are not limited to, Algiloy, nitinol, stainless steel, platinum, gold, titanium, other biocompatible metals, and combinations thereof. It should be understood that a preferred material for wireform sections 174 has an elasticity such that the material returns to its original shape after being deformed.

Leaflets 182 are supported by valve frame 172, as best seen in FIG. 11. Leaflets 182 may be made of suitable materials, including, but not limited to, bovine pericardium, equine pericardium, ovine pericardium, porcine aortic valve tissue, small intestinal submucosa (SIS), various biodegradable substrates for tissue engineered valves, and various relatively inert polymers, such as polyurethane. Wireform sections 174 may be covered with Dacron or other suitable medical grade covering, and leaflets 182 sewn to that covering. Alternatively, leaflets 182 may be attached directly to wireform sections 174 by appropriate means, such as sutures, clips, staples or other fastening devices.

Coupling elements 120 allow valve member 170 to be coupled and uncoupled from base member 130. As indicated above, recess 122 of coupling element 120 is dimensioned to receive tab 134 of mounting portion 132. Similarly, recess 138 of mounting portion 132 is dimensioned to receive tab 128 of coupling element 120. Valve member 170 is coupled and uncoupled from base 130 through engagement and disengagement of coupling element 120 and mounting portion 132.

It is contemplated that the two-piece (FIG. 3) and foldable (FIGS. 4-8) mechanical cardiovascular valves described above may also be configured for use as “exchangeable” valves in connection with a cardiovascular valve assembly that includes a permanent base member, such as disclosed in U.S. patent application Ser. No. 11/296,899 (filed Dec. 8, 2005, and published Jun. 22, 2006 as U.S. Patent Application Publication No. US2006/0136052,) and U.S. patent application Ser. No. 11/760,840 (filed Jun. 11, 2007), which are fully incorporated herein by reference. In this regard, the two-piece and foldable mechanical cardiovascular valves are engageable with a permanent base member, and can be conveniently removed from the base member to facilitate a valve exchange.

As indicated above, base member 130 may adapted to receive either a mechanical cardiovascular valve (FIGS. 9-10) or a bioprosthetic cardiovascular valve (FIGS. 11-13). Accordingly, it is contemplated that the base member of the present invention may be adapted for use with multiple different valve types (e.g., bioprosthetic and mechanical cardiovascular valves), thereby allowing a first type of cardiovascular valve to be attached to the base member, and later exchanged with a second type of cardiovascular valve. For example, a bioprosthetic cardiovascular valve can first be attached to the base member, and later exchanged for a mechanical cardiovascular valve, and vice-versa.

It should be appreciated that coupling elements 120 and mounting portions 132 as shown herein are for illustrating an embodiment of the present invention, and not for limiting same. In this regard, it is contemplated that other suitable interengagement means (including, but not limited to, depressions and/or protuberances) may be implemented to allow coupling and uncoupling of valve member and base member in accordance with the present invention.

It should also be appreciated that mechanical valve members described herein may be alternatively configured with single or multiple leaflets.

The foregoing description discloses specific embodiments of the present invention. It should be appreciated that these embodiments are described for purposes of illustration only, and that numerous alterations and modifications may be practiced by those skilled in the art without departing from the spirit and scope of the invention. It is intended that all such modifications and alterations be included insofar as they come within the scope of the invention as disclosed herein, and as claimed or the equivalents thereof.

1. A valve assembly comprising:
a mechanical valve member including:
a substantially U-shaped first valve body section;
a substantially U-shaped second valve body section, wherein said first valve body section is movable relative to said second valve body section; and
at least one leaflet pivotally mounted to each of said first and second valve body sections, each leaflet movable between an open position and a closed position.

2. A valve assembly according to claim 1, wherein said mechanical valve member further comprises:
first interengagement means for rotatably mounting said first valve body section to said second valve body section, said mechanical valve member is moveable between an unfolded position and a folded position by rotation of said first valve body section relative to said second valve body section.

3. A valve assembly according to claim 2, wherein said first interengagement means includes depressions and protuberances.

4. A valve assembly according to claim 2, wherein said valve assembly further comprises:
second interengagement means to allow said mechanical cardiovascular valve member to be coupled to and decoupled from a base member.

5. A valve assembly according to claim 4, wherein said second interengagement means includes depressions and protuberances.

6. A valve assembly according to claim 1, wherein said first and second valve body sections and said leaflets are formed of at least one of the following materials: pyrolytic carbon, stainless steel, nitinol, and polyurethane.

7. A valve assembly according to claim 1, wherein said valve assembly further comprises:
a base member including at least one mounting portion, the base member attachable to a tissue; and
said mechanical valve member further comprising:
at least one coupling element engageable with said at least one mounting portion to allow said valve member to be coupled to and decoupled from said base member.

8. A valve assembly according to claim 1, wherein said first valve body section is rotatably mounted to said second valve body section, said mechanical valve member moveable between an unfolded position and a folded position by rotation of said first valve body section relative to said second valve body section.

9. A valve assembly according to claim 1, wherein said first valve body section is separable from said second valve body section.

10. A valve assembly comprising:
a base member including at least one mounting portion, the base member attachable to a tissue; and
a bioprosthetic valve member including:
a valve frame comprising:
at least one wireform section, and
at least one coupling element engageable with said at least one mounting portion to allow said bioprosthetic valve member to be coupled to and decoupled from said base member; and
at least one leaflet mounted to said at least one wireform section.

11. A cardiovascular valve assembly according to claim 10, wherein said at least one leaflet is comprised of at least one of the following materials: bovine pericardium, equine pericardium, ovine pericardium, porcine aortic valve tissue, small intestinal submucosa (SIS), a biodegradable substrates for tissue engineered valves, and an inert polymer.

12. A valve assembly according to claim 1,
wherein each of said first and second valve body sections includes a respective front face, said respective front faces engaging each other when said first and second valve body sections are located adjacent to each other to form a mechanical cardiovascular valve.

13. A valve assembly according to claim 12, wherein each of said first and second valve body sections have respective sewing ring sections attached thereto for securing said first and second valve body sections to tissue of a heart.

14. A valve assembly according to claim 12, wherein said valve assembly further comprises:
interengagement means to allow said mechanical valve member to be coupled to and decoupled from a base member.

15. A valve assembly comprising:
a valve member including:
a substantially U-shaped first valve body section;
a substantially U-shaped second valve body section, wherein said first valve body section is moveable relative to said second valve body section; and
at least one leaflet pivotally mounted to each of said first and second valve body sections, each leaflet movable between an open position and a closed position.

16. A valve assembly according to claim 15, wherein said valve member further comprises:
first interengagement means for rotatably mounting said first valve body section to said second valve body section, said valve member is moveable between an unfolded position and a folded position by rotation of said first valve body section relative to said second valve body section.

17. A valve assembly according to claim 16, wherein said valve assembly further comprises:
second interengagement means to allow said valve member to be coupled to and decoupled from a base member.

18. A valve assembly according to claim 15, wherein said valve assembly further comprises:
a base member including at least one mounting portion, the base member attachable to a tissue; and
said valve member further comprising:
at least one coupling element engageable with said at least one mounting portion to allow said valve member to be coupled to and decoupled from said base member.

19. A valve assembly according to claim 15, wherein said first valve body section is separable from said second valve body section.

20. A valve assembly according to claim 15, wherein said valve member is a mechanical valve member.
